(19) United States
(12) Patent Application Publication
(10) Pub. No.: US 2005/0191672 A1
(43) Pub. Date: Sep. 1, 2005

(54) Antisense oligonucleotides and RNA-interfering molecules targeting PAK4

(75) Inventors: Ian James Popoff, Encinitas, CA (US); Todd Lee Van Arsdale, Carlsbad, CA (US)

Correspondence Address: AGOURON PHARMACEUTICALS, INC., 10777 SCIENCE CENTER DRIVE, SAN DIEGO, CA, 92121, US

(73) Assignee: AGOURON PHARMACEUTICALS, INC.

(21) Appl. No.: 11/045,182

(22) Filed: Jan. 27, 2005

Related U.S. Application Data
(60) Provisional application No. 60/542,571, filed on Feb. 6, 2004.

(51) Int. Cl. C12Q 1/68; C07H 21/02; A61K 48/00

(52) U.S. Cl. 435/6; 514/44; 536/23.1

(57) ABSTRACT

Compositions and methods for modulating the expression of the serine/threonine kinase PAK4 are provided. In particular, the invention relates to antisense compounds, particularly oligonucleotides and double-stranded RNA molecules, which specifically hybridize to nucleic acid molecules encoding PAK4. The oligonucleotides and RNA molecules decrease or inhibit PAK4 expression and thus, can be used in target identification and/or validation, to examine PAK4 pathways and the cellular effects of PAK4 expression, and to diagnose and/or treat abnormal cell growth or inflammation associated with PAK4 expression.

This application claims the benefit of U.S. Provisional Application No. 60/542,571 filed on Feb. 6, 2004, the contents of which is hereby incorporated by reference in its entirety.

### FIELD OF THE INVENTION

The present invention provides compositions and methods for modulating the expression of the serine/threonine kinase PAK4. In particular, the invention relates to antisense compounds, particularly oligonucleotides, and RNA-interfering molecules, which specifically hybridize to nucleic acid molecules encoding PAK4, and are thereby useful as PAK4 modulating agents.

### BACKGROUND OF THE INVENTION

The p21-activated protein kinase (PAK) family of serine/threonine protein kinases play an important role in cytoskeletal organization and cellular morphogenesis (Daniels et al., Trends Biochem. Sci. 24: 350-355 (1999); Sells et al., Trends Cell. Biol. 7:162-167 (1997)). PAK proteins were initially identified by their interaction with the active small GTPases, Cdc42, and Rac, and their shared sequence homology to yeast kinase Ste20 (Manser et al., Nature 367: 40-46 (1994)). In addition to mediating the regulation of actin cytoskeleton and cell adhesion by Cdc42 and Rac (Daniels et al., Trends Biochem. Sci. 24: 350-355 (1999)), some PAK proteins protect cells from apoptosis (Gnesutta et al., J. Biol. Chem. 276: 14414-14419 (2001); Rudel et al., Science 276: 1571-1574 (1997); Schurmann et al., Mol. Cell. Biol. 20: 453-461 (2000)); modulate mitogen activated protein (MAP) kinase pathways (Bagrodia et al., J. Biol. Chem. 270: 27995-27998 (1995); Brown et al., Curr. Biol. 6: 598-605 (1996); Chaudhary et al., Curr. Biol. 10: 551-554 (2000); Frost et al., EMBO J. 16: 6426-6438 (1997); King et al., Nature 396: 180-183 (1998); Sun et al., Curr. Biol. 10: 281-284 (2000)); mediate T-cell antigen receptor (TCR) signaling (Yablonski et al., EMBO J. 17: 5647-5657 (1998)); and respond to DNA damage (Roig et al., J. Biol. Chem. 274: 31119-31122 (1999)). Through these diverse functions, PAK proteins regulate cell proliferation and migration.

There are six known members of the PAK family divided into two subfamilies by their sequence similarity, namely PAK1-3 (PAK-1 subfamily) and PAK4-6 (PAK-II subfamily) (Dan et al., Trends Cell. Biol. 11: 220-230 (2001)). They share a conserved C-terminal kinase domain and a conserved Cdc42/Rac-interactive binding (CRIB) motif in the N-terminus. PAK1-3 have highly conserved sequences in these two regions across species. Several sequence differences between the subfamilies differentiate them in their cellular function and regulation. For example, in the kinase domain of PAK4-6, there is a serine (Ser) substitution (Ser445 in human PAK4) for an asparagine (Asn) (Asn395 in human PAK1) that is highly conserved in protein kinases (Hanks et al., Science 241: 42-52 (1998)). The side chain of this Asn residue is important in binding a metal ion that positions a phosphate group for transfer from ATP to the protein substrate (Bossemeyer et al., EMBO J. 12: 849-859 (1993)). Substitution by Ser, which has a shorter side chain, could affect the kinase activity. Indeed, replacement of Ser445 with Asn in human PAK4 generates a more active kinase (Qu et al., Mol. Cell. Biol. 21: 3523-3533 (2001)). In addition, the p21 binding domain (PBD) of PAK4-6 consists of a CRIB motif, but the surrounding regions are less conserved compared to those of PAK1-3. Unlike PAK1-3 which have similar interactions with the GTP-bound forms of Cdc42 and Rac, human PAK4, for example, prefers Cdc42 over Rac (Abo et al., EMBO J. 17: 6527-6540 (1998)). Moreover, PAK4 interacts with an effector loop mutant of Cdc42, suggesting that PAK4, at least, may play different roles than PAK1-3 (Abo et al., EMBO J. 17: 6527-6540 (1998); Lamarche et al., Cell 87: 519-529 (1996)).

The PBD of PAK1 contains a kinase inhibitory segment (KI, residues 138 to 147 of human PAK1) that interacts with the activation loop of PAK1 and thus, inhibits PAK1 kinase activity (Lei et al., Cell 102: 387-397 (2000)). The sequence of the PAK1 KI segment is not conserved in the PAK4-6 subfamily, suggesting that the latter subfamily may not have the similar auto-inhibition mechanism. Consistent with this hypothesis, active Cdc42 does not stimulate PAK4 autophosphorylation nor phosphorylation of substrates (Abo et al., EMBO J. 17: 6527-6540 (1998)).

It is known that PAK4 is recruited to the Golgi apparatus by activated Cdc42 (Abo et al., EMBO J. 17: 6527-6540 (1998)). Phosphorylation of the activation loop may activate PAK4 kinase activity since substitution of Ser474 in the activation loop of PAK4 with Glu increases PAK4 kinase activity (Qu et al., Mol. Cell. Biol. 21: 3523-3533 (2001)). PAK4 kinase activity induces localized actin polymerization and filopodia formation (Abo et al., EMBO J. 17: 6527-6540 (1998)), as well as anchorage-independent cell growth (Qu et al., Mol. Cell. Biol. 21: 3523-3533 (2001)). In addition, PAK4 phosphorylates the pro-apoptotic protein BAD and protects cells from apoptosis. The ability of PAK4 to interact with the effector loop mutant of Cdc42 also suggests that PAK4 plays a role not attributed to other PAK proteins in Cdc42-mediated cytoskeleton organization (Abo et al., EMBO J. 17: 6527-6540 (1998)).

The full-length PAK4 nucleic acid and amino acid sequences are disclosed in U.S. Pat. No. 6,013,500, and have been deposited in GenBank under accession numbers AF005046 (mRNA) and MD01210 (amino acid). Sequencing of the PAK4 gene revealed an N-terminal regulatory domain (GBD/CRIB domain) and a C-terminal kinase domain similar to other PAK proteins (Abo et al., EMBO J. 17: 6527-6540 (1998)). Modulation of human PAK4 activity is reported to result in alterations in cellular processes affecting cell growth and adhesion. For example, overexpression of PAK4 in fibroblasts leads to morphological changes that are characteristic of oncogenic transformation through induction of anchorage-independent growth and inhibition of apoptosis (Gnesutta et al., J. Biol. Chem. 276:14414-14419 (2001); Qu et al., Mol. Cell. Biol. 21: 3523-2533 (2001)).

Neoplastic cells, due to their inherent genetic instability, have lost many of the control mechanisms regulating cell division and, therefore, neoplastic cells are more susceptible to cell-cycle modulation or intervention as a means of inducing cell death. Further, because alterations in cellular growth is one of the differences between normal cells and cancer cells, proteins involved in cellular growth are attractive targets for developing agents effective for use in diagnosing and treating cell proliferative disorders. One such target is PAK4. Thus, there is a need in the art for tools useful in studying the role of PAK4 and PAK4 pathways involved in normal and abnormal cell growth, as well as for tools useful in identifying inhibitors of molecular targets such as PAK4, and in identifying and/or confirming the action of PAK4 modulatory compounds.

### SUMMARY OF THE INVENTION

The present invention is directed to antisense and RNA-interfering compounds, particularly oligonucleotides and double-stranded RNA molecules (siRNAs), which target nucleic acid molecules encoding PAK4 and modulate the expression of PAK4. The antisense oligonucleotides and/or RNA-interfering molecules can be used in target identification and/or validation studies as well as to examine the effects of PAK4 expression on cells and to study PAK4 pathways.

Pharmaceutical and other compositions comprising the antisense and/or RNA-interfering compounds of the invention are also provided. Further provided are methods of modulating the expression of PAK4 as well as methods of diagnosing, treating, or preventing a disease or disorder associated with abnormal expression of PAK4.

More specifically, the present invention provides antisense oligonucleotides comprising about 8 to about 50 nucleic acid bases in length targeted to a nucleic acid molecule encoding PAK4, wherein the antisense oligonucleotides specifically hybridize to and decrease or inhibit the expression of PAK4. In one aspect of the present invention, PAK4 is preferably human PAK4 having the sequence set forth in SEQ ID NO:1. In another aspect, PAK4 is a mutant form of PAK4.

Preferably, the antisense oligonucleotides comprise about 8 to about 30 nucleic acid bases in length. Even more preferably, the antisense oligonucleotides are about 20 nucleic acid bases in length. In the most preferred embodiments, the antisense oligonucleotides have the sequence set forth in SEQ ID NO:2, 3, 4, 5, 6, 7, 8, 9, 10, 11, 12, 13, 14, 15, 16, 17, 18, 19, 20, 21, 22, 23, 24, 25, 26, 27, or 28.

The antisense oligonucleotides can include at least one modified internucleoside linkage, such as, for example, a phosphorothioate linkage. In other embodiments, the antisense oligonucleotides can include at least one modified sugar moiety, such as, for example, 2′-O-methoxymethyl sugar moiety. It is further contemplated that the antisense oligonucleotides can include a modified nucleic acid base, such as, for example, 5-methylcytosine.

The present invention also relates to ribonucleic acid-(RNA)-interfering molecules comprising a short, double-stranded RNA molecule or siRNA. One strand of the RNA molecule is a ribonucleotide sequence that corresponds to a nucleotide sequence encoding PAK4. The second strand is a ribonucleotide sequence that is complementary to the first strand. The double-stranded RNA-interfering molecule decreases or inhibits the expression of PAK4.

More specifically, the strands of the RNA-interfering molecule preferably comprise from about 8 to about 30 nucleic acid bases in length. More preferably, the strands are about 21 nucleic acid bases in length. Most preferably, the first ribonucleotide sequence is a sequence selected from the group consisting of: SEQ ID NOS:29, 31, and 33, and the second ribonucleotide sequence is a sequence selected from the group consisting of: SEQ ID NOS:30, 32, and 34.

The present invention also relates to compositions comprising at least one antisense oligonucleotide and a pharmaceutically acceptable carrier, diluent, or salt, wherein the antisense oligonucleotide is targeted to a nucleic acid molecule encoding PAK4 and wherein the antisense oligonucleotide specifically hybridizes to and decreases or inhibits the expression of PAK4. In other embodiments, the compositions comprise at least one RNA-interfering molecule and a pharmaceutically acceptable carrier, diluent, or salt, wherein the RNA-interfering molecule is targeted to a nucleic acid molecule encoding PAK4 and wherein the RNA-interfering molecule specifically hybridizes to and decreases or inhibits the expression of PAK4.

In other aspects of the present invention, the compositions further comprise at least one agent useful in the treatment of abnormal cell growth, wherein the compound is not an antisense oligonucleotide or an RNA-interfering molecule, such as, for example, mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxic compounds, anti-hormones, or anti-androgens. In still other aspects of the present invention, the compositions comprise at least one antisense oligonucleotide in combination with at least one RNA-interfering molecule.

The present invention also relates to methods of decreasing or inhibiting the expression of PAK4 in cells or tissues comprising, in one embodiment, contacting the cells or tissues with at least one antisense oligonucleotide and/or RNA-interfering molecule of the invention so that expression of PAK4 is decreased or inhibited. In preferred embodiments of the invention, the cells or tissues are human cells or tissues and the antisense oligonucleotide and/or RNA-interfering molecule is a component of a composition comprising a pharmaceutically acceptable salt, diluent, or carrier.

In still other aspects of the invention, methods of diagnosing and/or treating a human having or suspected of having a disease or condition associated with abnormal PAK4 expression are provided, comprising administering a therapeutically effective amount of at least one antisense oligonucleotide or RNA-interfering molecule disclosed herein so that expression of PAK4 is decreased or inhibited. The disease or condition includes, but is not limited to, abnormal cell growth, such as, for example, cancer, benign proliferative disease, psoriasis, benign prostatic hypertrophy, or restinosis. In still other aspects, the disease or condition is an inflammatory disease or condition, such as, for example, an autoimmune disease, cell-mediated rejection, graft-versus-host disease, or arthritis.

### BRIEF DESCRIPTION OF THE DRAWINGS

FIG. 1 is a graph depicting PAK4 expression in A549 cells 24 hours after treatment with PAK4 antisense oligonucleotides of the present invention.

FIGS. 2A and 2B are bar graphs depicting PAK4 RNA expression in A549 cells 24 hours (A) and 48 hours (B) after treatment with PAK4 siRNAs of the present invention.

FIG. 3 is a bar graph depicting PAK4 RNA expression in A549 cells 48 hours after treatment with PAK4 siRNAs of the present invention. FIG. 3 demonstrates that the suppressive effects of the siRNAs on PAK4 expression are time-dependent. In this Figure, C is the control siRNA; P is the Pak4-siRNA. The designations C1 and C2 are replicate samples; P1 and P2 are replicate samples. Each was split into two aliquots for RNA prep; each RNA was used for two PCR reactions; P ave is the average of all PCR quantities for P PCRs divided by C ave (100%); C ave is the average of all PCR quantities, defined as 100%.

### DETAILED DESCRIPTION OF THE INVENTION

The present invention is directed to antisense oligonucleotides and RNA-interfering molecules, siRNAs, which target, i.e., hybridize to, nucleic acid molecules encoding PAK4 and modulate, preferably, decrease or inhibit, PAK4 expression. Thus, the present invention is further directed to compositions and methods for modulating PAK4 expression, as well as to compositions and methods for target identification and/or validation, and to compositions and methods for diagnosing and/or treating diseases or conditions associated with abnormal expression of PAK4.

Definitions

As used herein, the term “nucleic acid” encompasses DNA encoding PAK4, RNA (including, but not limited to, pre-mRNA and mRNA) transcribed from such DNA, and cDNA derived from such RNA, unless specifically indicated otherwise. “Ribonucleic acid” refers to a RNA-specific nucleic acid.

As used herein, the term “antisense” encompasses interference with normal nucleic acid function(s) as caused by the specific hybridization of an oligomeric compound to its target nucleic acid. The function(s) interfered with include, but is not limited to, replication, transcription, translocation of RNA to the site of protein translation, translation of protein from RNA, splicing of RNA to yield one or more mRNA species, and catalytic activity which may be engaged in or facilitated by RNA.

The terms “hybridization” and “complementary”, as used herein, refer to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary or hybridizable to each other at that position. The oligonucleotide and the DNA or RNA hybridize when a sufficient number of corresponding positions in each molecule are occupied by nucleotides which can hydrogen bond with each other. It is understood in the art that the sequence of an antisense oligonucleotide or an RNA-interfering molecule need not be 100% complementary to that of its target nucleic acid to hybridize thereto. An antisense oligonucleotide or an RNA-interfering molecule are specifically hybridizable when binding of the compound to the target DNA or RNA molecule interferes with the normal function of the target DNA or RNA, and there is a sufficient degree of complementarity to avoid non-specific binding of the antisense oligonucleotide or RNA-interfering molecule to non-target sequences under conditions in which specific binding is desired, e.g., under physiological conditions in the case of in vivo assays or therapeutic treatment, or, in the case of in vitro assays, under conditions in which the assays are performed.

The term “oligonucleotide” refers to an oligomer or polymer of ribonucleic acid (RNA) or deoxyribonucleic acid (DNA) or mimetics thereof. This term includes, but is not limited to, oligonucleotides composed of naturally occurring and/or synthetic nucleic acid bases, sugars, and covalent internucleoside (backbone) linkages, as well as oligonucleotides having non-naturally occurring portions which function similarly. Such modified or substituted oligonucleotides are often preferred over native forms because of desirable properties, such as, for example, enhanced cellular uptake, enhanced affinity for nucleic acid targets, and/or increased stability in the presence of nucleases.

As used herein, the term “antisense oligonucleotide” refers to synthetic oligonucleotides which bind complementary nucleic acids (i.e. sense strand sequences) via hydrogen bonding, thus inhibiting translation of these sequences.

The term “RNA interference” or “RNAi” refers to a method to suppress, inhibit, or decrease gene expression in a cell through the use of a “double-stranded RNA” (“dsRNA”) molecule, whereby at least one strand of the dsRNA binds to the mRNA of the targeted gene and prevents the expression of that gene and the production of the gene product. “siRNA,” “small-interfering RNA,” or “RNA-interfering molecule” are alternative terms referring to any of the small (less than about 30 nucleic acid bases in length) double-stranded RNA molecules used in the present invention.

PAK4 Antisense Oligonucleotides

The cDNA sequence of human PAK4, from which the preferred oligonucleotides disclosed herein were deduced, is set forth in SEQ ID NO:1 (GenBank Accession No. AF005046). The oligonucleotides of the invention are complementary to at least a portion of the nucleotide sequence encoding human PAK4. Thus, the antisense oligonucleotides bind in a sequence-specific manner to the target mRNA, i.e., PAK4 mRNA. This binding may reduce or inhibit the ability of the mRNA to be translated to protein, or cause RNAse-mediated degradation of Pak4 RNA, and thus, interferes with the normal production and biological activity of PAK4. The selective reduction or inhibition of mRNA coding for PAK4 allows for the study of PAK4 pathways and thus, for the identification and/or validation of target molecules involved with the pathways. More specifically, PAK4 pathways and cellular effects of PAK4 expression can be analyzed by correlating reduced or inhibited PAK4 expression with changes in cellular phenotype. Preferably, the antisense oligonucleotides are about 8 to about 50 nucleic acid bases in length, more preferably, about 8 to about 30 nucleic acid bases in length, and, even more preferably, about 20 nucleic acid bases in length. The most preferred antisense oligonucleotides are set forth in Table 1.

TABLE 1

PAK4 Antisense Oligonucleotides

| | | | |
|---|---|---|---|
| | | Alignment | |
| | | with SEQ ID | SEQ |
| Position | Sequence | NO: 1 (nt | ID |
| Name | (5′-3′) | residues) | NO: |

| | | | |
|---|---|---|---|
| PAK4-1 | gacgaattccaccacactgg | 1 | 2 |
| PAK4-21 | cttgcaccgccaccaccgcg | 21 | 3 |
| PAK4-51 | actccgcgccctcgcgcctc | 51 | 4 |
| PAK4-81 | gtcgctcgcggcctaactgc | 81 | 5 |
| PAK4-111 | cttcgggttactcatcggct | 111 | 6 |
| PAK4-161 | atgctggtgggacagaagtg | 161 | 7 |
| PAK4-331 | gccgactcctcgatcaggct | 331 | 8 |
| PAK4-501 | tgtctctccgcagggagttg | 501 | 9 |
| PAK4-811 | gtgttaaagggccggccagc | 811 | 10 |
| PAK4-1151 | gtaggagcgggggtcgcctg | 1151 | 11 |
| PAK4-1261 | tgcttgcgcaggtccatctt | 1261 | 12 |
| PAK4-1391 | tccttccaggaactccatga | 1391 | 13 |
| PAK4-1561 | gacagcttcaccctgccatc | 1561 | 14 |
| PAK4-1871 | ccgctgggcagggtctcgca | 1871 | 15 |
| PAK4-2071 | gcatctcccgggctgggagg | 2071 | 16 |
| PAK4-2131 | aactggagttcagtagtagg | 2131 | 17 |
| PAK4-2191 | tcctgggagcctcgcttgct | 2191 | 18 |
| PAK4-2241 | ttcctggagacagaagaaca | 2241 | 19 |
| PAK4-2331 | gcacacactcatacatgttc | 2331 | 20 |
| PAK4-2351 | acatgcacactcacacgcgt | 2351 | 21 |
| PAK4-2426 | ctgggtgtcaggcaaggcgc | 2426 | 22 |
| PAK4-2525 | tccccatccagccacagaaa | 2525 | 23 |
| PAK4-2581 | aggtgcagtagtcatttgct | 2581 | 24 |
| PAK4-2665 | caggacagggaccatctgtc | 2665 | 25 |
| PAK4-2701 | gcagtggttctgccagggcc | 2701 | 26 |
| PAK4-2734 | ctgcgctgaccgggcaggaa | 2734 | 27 |
| PAK4-2765 | ctaactcgaggcaggggtgg | 2765 | 28 |

The antisense oligonucleotides are suitable for numerous in vitro and in vivo applications including, but not limited to, target validation and proof-of-principle studies to examine the cellular effects of PAK4. General target validation and proof-of-principle protocols are well known in the art.

In addition, the antisense oligonucleotides of the invention can be used to study the PAK4 pathways and the cellular effects of PAK4 reduction or inhibition by comparing phenotype or other measurement of PAK4 expression in cells with normal PAK4 expression to cells with abnormal PAK4 expression induced by the inventive antisense oligonucleotides. Moreover, the inventive antisense oligonucleotides can be used to diagnose and/or treat abnormal proliferative states in cells or tissues suspected of having a disease or condition associated with abnormal cell growth associated with PAK4 expression or a PAK4 pathway. Such diseases or conditions include, but are not limited to, cancer, benign proliferative disease, psoriasis, benign prostatic hypertrophy, and restinosis. In addition, the inventive antisense oligonucleotides can be used to treat an inflammatory disease or condition, such as, for example, an autoimmune disease, cell-mediated rejection, graft-versus-host disease, or arthritis associated with abnormal PAK4 expression or abnormal PAK4 pathways. In such methods, normal cellular PAK4 expression is preferably a reference against which the reduced or inhibited cellular PAK4 expression is compared. The reference cells can be optionally treated with one or more functional oligonucleotides that do not affect PAK4 expression.

PAK4 Antisense Oligonucleotide Modifications

As noted above, the most preferred antisense oligonucleotides are set forth in Table 1. However, the inventive antisense oligonucleotides are not limited to the sequences of Table 1, but include any antisense oligonucleotide sequence having the ability to bind to human PAK4 and decrease or inhibit the expression thereof. Such PAK4 antisense oligonucleotides include, but are not limited to, oligonucleotides containing modified backbones or non-natural internucleoside linkages. Oligonucleotides having modified backbones include, but are not limited to, those that retain a phosphorus atom in the backbone and those that do not have a phosphorus atom in the backbone, i.e., oligonucleosides.

Preferred modified oligonucleotide backbones include, but are not limited to, phosphorothioates, chiral phosphorothioates, phosphorodithioates, phosphotriesters, aminoalkylphosphotriesters, methyl and other alkyl phosphonates including, but not limited to, 3′-alkylene phosphonates and chiral phosphonates, phosphinates, phosphoramidates including, but not limited to, 3′-amino phosphoramidates and aminoalkylphosphoramidates, thionophosphoramidates, thionoalkylphosphonates, thionoalkylphosphotriesters, and boranophosphates. The oligonucleotide backbones have normal 3′-5′ linkages, are 2′-5′ linked analogs, or have inverted polarity wherein the adjacent pairs of nucleoside units are linked 3′-5′ to 5′-3′ or 2′-5′ to 5′-2′. Exemplary oligonucleotides mimetics are oligonucleotides with phosphorothioate backbones and oligonucleotides with heteroatom backbones, and in particular, —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂—, and—O—N(CH₃)—CH₂—CH₂—, wherein the native phosphodiester backbone is represented as —O—P—O—CH₂—. See, e.g., U.S. Pat. No. 5,489,677, U.S. Pat. No. 5,602,240, and U.S. Pat. No. 5,034,506. Most preferred backbones are 3′-5′ linked phosphorothioates.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include, but are not limited to, oligonucleotides having morpholino linkages (formed in part from the sugar portion of a nucleoside); siloxane backbones; sulfide, sulfoxide, and sulfone backbones; formacetyl and thioformacetyl backbones; methylene formacetyl and thioformacetyl backbones; alkene-containing backbones; sulfamate backbones; methyleneimino and methylenehydrazino backbones; sulfonate and sulfonamide backbones; amide backbones; and others having mixed N, O, S, and CH₂ components.

In alternative PAK4 oligonucleotide mimetics, both the sugar and the internucleoside linkage, i.e., the backbone, of the nucleotide units are replaced with novel groups. The base units are maintained for hybridization with an appropriate PAK4 nucleic acid target. One such oligomeric compound, an oligonucleotide mimetic that has been shown to have excellent hybridization properties, is a peptide nucleic acid (PNA). In PNA compounds, the sugar-backbone of the oligonucleotide is replaced with an amide-containing backbone, in particular, an aminoethylglycine backbone. The nucleic acid bases are retained and are bound directly or indirectly to aza nitrogen atoms of the amide portion of the backbone.

Modified oligonucleotides may also contain one or more substituted sugar moieties. For example, oligonucleotides may comprise one of the following at the 2′ position: OH; F; O-, S-, or N-alkyl; O-, S-, or N-alkenyl; O-, S-, or N-alkynyl; or O-alkyl-O-alkyl, wherein the alkyl, alkenyl, and alkynyl may be substituted or unsubstituted C₁ to C₁₀ alkyl, C₂ to C₁₀ alkenyl, and C₂ to C₁₀ alkynyl, respectively. More specifically, the modified sugar moieties may be, for example, O((CH₂)ₙO)ₘCH₃, O(CH₂)ₙOCH₃, O(CH₂)ₙNH₂, O(CH₂)ₙCH₃, O(CH₂)ₙNH₂, and O(CH₂)ₙON((CH₂)ₙCH₃))₂, where n and m are from about zero (0) to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of the oligonucleotide, or a group for improving the pharmacodynamic properties of the oligonucleotide. Oligonucleotides may also have sugar mimetics, such as, for example, cyclobutyl moieties in place of the pentofuranosyl sugar.

A 2′ modification motif of 5 modified/10 unmodified/5 modified, 4/12/4, or 3/14/3, for example, has been accepted to be more stable than unmodified in plasma and in vivo. This modification known as “gap+wings,” still promotes RNAseH-mediated cleavage of target RNA (gap, middle section of un-modified sugar on the DNA, while the “Wings,” or ends, provide exonuclease stability) (see U.S. Pat. Nos. 5,576,208, 5,859,221, and 5,872,232).

Oligonucleotides may also include nucleic acid base (often referred to in the art simply as “base”) modifications or substitutions. As used herein, “unmodified” or “natural” nucleic acid bases include the purine bases adenine (A) and guanine (G), and the pyrimidine bases thymine (T), cytosine (C), and uracil (U). Modified nucleic acid bases include, but are not limited to, synthetic and natural nucleic acid bases, such as, for example, 5-methylcytosine (5-me-C); 5-hydroxymethylcytosine; xanthine; hypoxanthine; 2-aminoadenine; 6-methyl and other alkyl derivatives of adenine and guanine; 2-propyl and other alkyl derivatives of adenine and guanine; 2-thiouracil; 2-thiothymine; 2-thiocytosine; 5-halouracil and 5-halocytosine; 5-propynyluracil and 5-propynylcytosine; 6-azouracil, 6-azocytosine, and 6-azothymine; 5-uracil (pseudouracil); 4-thiouracil; 8-halo, 8-amino, 8-thiol, 8-thioalkyl, 8-hydroxyl, and other 8-substituted adenines and guanines; 5-halo, particularly, 5-bromo, 5-trifluoromethyl, and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine.

Certain nucleic acid bases are particularly useful for increasing the binding affinity of the inventive antisense oligonucleotides. These include, but are not limited to, 5-substituted pyrimidines, 6-azapyrimidines, and N-2, N-6, and 0-6 substituted purines, including, but not limited to, 2-aminopropyladenine, 5-propynyluracil, and 5-propynylcytosine. 5-methylcytosine substitutions have been shown to increase nucleic acid duplex stability by 0.6-1.2° C. (Sanghvi et al. (eds.), Antisense Research and Applications, CRC Press, Boca Raton, pp. 276-278 (1993)) and are presently preferred base substitutions, even more particularly when combined with 2′-O-methoxyethyl sugar modifications.

Another modification of the PAK4 antisense oligonucleotides of the invention involves chemically linking the oligonucleotide and one or more moieties which enhance the activity, cellular distribution, cellular uptake, and/or binding affinity of the oligonucleotide. Such moieties include, but are not limited to, lipid moieties, such as, for example, a cholesterol moiety cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethyl-ammonium 1,2-di-O-hexadecyl-rac-glycero-3-H-phosphonate, a polyamine, or a polyethylene glycol chain, or adamantine acetic acid, a palmityl moiety, or an octadecylamine or hexylamino-carbonyl-oxycholesterol moiety. The resulting “chimeric” antisense oligonucleotides are antisense oligonucleotides which contain two or more chemically distinct regions, e.g., an oligonucleotide and a chemical moiety. Chimeric antisense compounds of the invention may be formed as composite structures of two or more oligonucleotides, modified oligonucleotides, oligonucleosides, and/or oligonucleotide mimetics as described above. Such compounds have also been referred to in the art as hybrids, gapmers, chimeras, and fusion proteins.

For all modifications, it is not necessary for every position in a given oligonucleotide to be uniformly modified. In addition, more than one modification may be incorporated into a single oligonucleotide or a single nucleoside within an oligonucleotide.

Synthesis of the PAK4 Antisense Oligonucleotides and Cellular Transfection

The PAK4 antisense oligonucleotides may be routinely made in vitro through the well-known technique of solid phase synthesis (see, for example, Gait, “An Introduction To Modern Methods of DNA Synthesis,” Oligonucleotide Synthesis a Practical Approach, Gait (ed.), IRL Press, Oxford, UK, pp. 1-22 (1984); Gait et al., “Solid-Phase Synthesis of Oligodeoxyribonucleotides by the Phosphotriester Method,” Ibid., pp. 83-116). Other means, e.g., chemical and/or enzymatic, for such synthesis known in the art may additionally or alternatively be employed. See, e.g., Brown et al., “Modern Machine-Aided Methods of Oligonucleotide Synthesis,” Oligonucleotides and Analogues a Practical Approach, Eckstien (ed.), IRL Press, Oxford, UK (1995); Au et al., Biochem. Biophys. Res. Commun. 248(1): 200-203 (1998)). Equipment for such synthesis is available from several vendors including, but not limited to, Applied Biosystems (Foster City, Calif.). Alternatively, the antisense oligonucleotides may be synthesized by a cellular RNA polymerase or a bacteriophage RNA polymerase, such as, for example, T3, T7, or SP6.

When synthesized in vitro, the antisense oligonucleotides may be purified prior to introduction into the host cell. For example, the oligonucleotides may be purified from a mixture by extraction with a solvent or resin, by precipitation, by electrophoresis, by chromatography, or by a combination thereof. The oligonucleotides may be dried for storage or dissolved in an aqueous solution which may contain buffers or salts for stabilization.

Antisense sequences targeting PAK4 are transfected into suitable host cells by methods known in the art. Alternatively, host cells can be electroporated in suspension, following the instructions of the manufacturer of the electroporation device. The term “host cells” includes, but is not limited to, any progeny of the subject host cells. It is understood that all progeny may not be identical to the parental cell since there may be mutations that occur during replication. However, such progeny are included when the term “host cells” is used. Host cells include, but are not limited to, microbial, yeast, insect, and mammalian cells. Preferred host cells include human lung adenocarcinoma A549, or human breast epithelial adenocarcinoma MCF7.

RNA-Interference Molecules

RNA-interference (RNAi) is a method for post-transcriptional gene silencing by double-stranded RNA (dsRNA) (Montgomery et al., Trends Genet. 14: 255-258 (1998); Fire, Trends Genet 15: 358-363 (1999); Hunter Curr. Biol. 9: R440-442 (1999); Sharp, Genes & Dev. 13: 139-141 (1999); Harborth et al., J. Cell Sci. 114: 4557-4565 (2001); Masters et al., Proc. Natl. Acad. Sci. USA 98: 8012-8017 (2001)). The hallmark of RNAi is its specificity. The dsRNA reduces expression of the gene from which the dsRNA sequence is derived, without a detectable effect on the expression of genes unrelated in sequence (Fire et al., Nature 391: 806-811 (1998)). Thus, RNAi is a suitable technique for numerous in vitro and in vivo applications including, but not limited to, target validation and proof-of-principle studies to examine the cellular effects of PAK4 expression or the effects of a given compound on PAK4 expression. In addition, the siRNAs of the invention can be used to study the PAK4 pathways and the cellular effects of PAK4 reduction or inhibition by comparing phenotype or other measurement of PAK4 expression in cells with normal PAK4 expression to cells with abnormal PAK4 expression induced by the inventive siRNAs. Moreover, the inventive siRNAs can be used to diagnose and/or treat abnormal proliferative states in cells or tissues suspected of having a disease or condition associated with abnormal cell growth associated with PAK4 expression or a PAK4 pathway. Such diseases or conditions include, but are not limited to, cancer, benign proliferative disease, psoriasis, benign prostatic hypertrophy, and restinosis.

In addition, the inventive siRNAs can be used to treat an inflammatory disease or condition, such as, for example, an autoimmune disease, cell-mediated rejection, graft-versus-host disease, or arthritis associated with abnormal PAK4 expression or abnormal PAK4 pathways. In such methods, normal cellular PAK4 expression is preferably a reference against which the reduced or inhibited cellular PAK4 expression is compared. The reference cells can be optionally treated with one or more functional siRNA, or other nucleic acid molecule, that does not affect PAK4 expression.

The cDNA sequence of human PAK4, from which the preferred siRNAs disclosed herein were deduced, is set forth in SEQ ID NO:1 (GenBank Accession No. AF005046). The siRNAs bind in a sequence-specific manner to the target mRNA, i.e., PAK4 mRNA. This binding may reduce or inhibit the ability of the mRNA to be translated to protein, or cause RSC-complex-mediated degradation of PAK4 RNA, and thus, interferes with the normal production and biological activity of PAK4. The selective reduction or inhibition of mRNA coding for PAK4 allows for the study of PAK4 pathways and thus, for the identification and/or validation of target molecules involved with the pathways. More specifically, PAK4 pathways and cellular effects of PAK4 expression can be analyzed by correlating reduced or inhibited PAK4 expression with changes in cellular phenotype. Preferably, the siRNAs are about 8 to about 30 nucleic acid bases in length (per strand), more preferably, about 15 to about 25 nucleic acid bases in length (per strand), and, even more preferably, about 21 nucleic acid bases in length (per strand). The most preferred siRNA molecules are set forth in Table 2.

TABLE 2

sIRNA Sequences

| | | | | |
|---|---|---|---|---|
| | | | | Alignment |
| | | | | with SEQ |
| | | | Antisense siRNA | ID NO: 1 |
| Position | Target Sequences | Sense RNA Strand | Strand | (nt |
| Name | (5′-3′) | (5′-3′) | (5′-3′) | residues) |

| | | | | |
|---|---|---|---|---|
| PK4-481 | aacatgtcggtgacacgctcc | caugucggugacacgcucctt | ggagcgugucaccgaca | 301 |
| | (SEQ ID NO: 41) | (SEQ ID NO: 29) | ugtt | |
| | | | (SEQ ID NO: 30) | |
| PK4-1534 | aagagcgactcgatcctgctg | gagcgacucgauccugcugtt | cagcaggaucgagucgc | 1354 |
| | (SEQ ID NO: 42) | (SEQ ID NO: 31) | uctt | |
| | | | (SEQ ID NO: 32) | |
| PK4-1819 | aacctgcacaaggtgtcgcc | ccugcacaaggugucgccatt | uggcgacaccuugugca | 1639 |
| | a | (SEQ ID NO: 33) | ggtt | |
| | (SEQ ID NO: 43) | | (SEQ ID NO: 34) | |
| PK4-1 | aactcgccaatcttgatgaag | cuucaucaagauuggcgagtt | cucgccaaucuugauga | 1179 |
| | (SEQ ID NO: 44) | (SEQ ID NO: 45) | agtt | |
| | | | (SEQ ID NO: 46) | |

The length of the siRNA affects the silencing efficiency, i.e., the ability to inhibit or reduce PAK4 expression, of the molecule (Tuschl et al., Genes & Dev. 13: 3191-3197 (1999); Elbashir et al., Nature 411: 494-498 (2001); Elbashir et al., Genes & Dev. 15: 188-200 (2001)). The most efficient silencing typically is obtained with siRNAs composed of approximately 21 nucleotides per strand, paired in a manner to have a 2-nucleotide overhang at the 3′ end. The sequence of the 3′ overhang also makes a contribution to the specificity of target recognition restricted to the unpaired nucleotide adjacent to the first base pair.

Synthesis of the PAK4 siRNAs and Cellular Transfection

The siRNAs may be synthesized either in vivo or in vitro and PAK4 expression may be targeted by specific transcription of the siRNAs into an organ, tissue, or cell, for example. The siRNA strands may be polyadenylated and/or may be capable of being translated into a polypeptide by a cell's translational apparatus. Alternatively, host cells can be electroporated in suspension, following the instructions of the manufacturer of the electroporation device.

In vitro, siRNAs may be chemically or enzymatically synthesized by manual or automated reactions (Dykxhoorn et al., Nature Reviews 4: 457-467 (2003)). For example, the inventive siRNAs can be chemically synthesized using protected ribonucleoside phosphoramidites and a DNA/RNA synthesizer. Alternatively, the inventive siRNAs may be synthesized by a cellular RNA polymerase or a bacteriophage RNA polymerase, such as, for example, T3, T7, or SP6. See, e.g., Sui et al., PNAS USA 99: 5515-5520 (2002); Brummelkamp et al., Science 296: 550-553 (2002); Paul et al., Nature Biotechnology 20: 505-508 (2002); Lee et al., Nature Biotechnology 20: 500-505 (2002); and Castanotto et al., RNA 8:1454-1460 (2002).

In vivo, siRNAs may be expressed from a transgene or expression construct via transfection into suitable host cells, including, but not limited to, cells in which a vector can be propagated and its DNA expressed. Host cells include, but are not limited to, microbial, yeast, insect, and mammalian cells, such as, for example, human immortalized cell lines.

Methods of stable transfection are well known in the art. For example, the inventive siRNAs can be inserted into a recombinant expression vector, e.g., a plasmid, virus, or other vehicle known in the art that has been manipulated by insertion or incorporation of at least one PAK4 siRNA. Such expression vectors preferably contain a regulatory region, such as, for example, promoter, enhancer, silencer, splice donor and acceptor, and/or polyadenylation sequences which may be used to transcribe the RNA strand (or strands). Such regulators are well known in the art. The expression vector typically contains an origin of replication along with a promoter and, optionally, specific genes which allow phenotypic selection of the transformed cells. One skilled in the art would be able to readily determine which vectors are suitable for use in the present invention. Examples of suitable vectors include, but are not limited to pSuppressor Retro (Imgenex Corp., San Diego, Calif.), pSuppressorAdeno (Imgenex Corp.), and pShuttle-H1 (ClonTech).

If synthesized chemically or by in vitro enzymatic synthesis, the siRNAs may be purified prior to introduction into the host cell. For example, siRNAs may be purified from a mixture by extraction with a solvent or resin, by precipitation, by electrophoresis, by chromatography, or by a combination thereof. The siRNAs may be dried for storage or dissolved in an aqueous solution optionally containing, for example, buffers or salts to promote annealing and/or stabilization of the duplex strands.

siRNA Modifications

The siRNA may comprise one or more strands of polymerized ribonucleotide, optionally including modifications to either the phosphate-sugar backbone or the nucleoside. Modifications include, but are not limited to, substituting dT for U at the 3′ end, and adding an acid-labile orthoester group, such as, for example, bis(acetoxyethoxy)-orthoformate (2′-ACE), at the 2′ position to protect the RNA.

The double-stranded structure may be formed by a single self-complementary RNA strand or two complementary RNA strands. siRNA duplex formation may be initiated either inside or outside the cell. The RNA may be introduced in an amount which allows delivery of at least one copy per cell. Higher doses of double-stranded material may yield more effective inhibition. Inhibition is sequence-specific in that nucleotide sequences corresponding to the duplex region of the RNA are targeted for genetic inhibition. RNA containing a nucleotide sequence(s) identical to a portion of the target gene is preferred for inhibition. Also, siRNA sequences with insertions, deletions, and/or single point mutations relative to the target sequence have been found to be effective for inhibition. Thus, sequence identity may optimized by using alignment algorithms known in the art and calculating the percent difference between the nucleotide sequences.

Alternatively, the duplex region of the siRNA may be defined functionally as a nucleotide sequence that is capable of hybridizing with a portion of the target gene transcript.

PAK4 Expression

Modulation of PAK4 expression can be assayed in a variety of ways known in the art. As non-limiting examples, PAK4 mRNA levels can be quantitated by Northern blot analysis, competitive polymerase chain reaction (PCR), Reverse Transcriptase-PCR (RT-PCR), or variations thereof, which are all well known in the art. RNA analysis can be performed on total cellular RNA or poly(A)+ mRNA. Methods of RNA isolation are disclosed in, for example, Ausubel et al., Current Protocols in Molecular Biology, Vol. 1, pp. 4.1.1-4.2.9 and 4.5.1-4.5.3, John Wiley & Sons, Inc. (1993).

Similarly, protein levels of PAK4 can be quantitated in a variety of ways well-known in the art, such as, for example, immunoprecipitation, Western blot analysis (immunoblotting), ELISA, or fluorescence-activated cell sorting (FACS). Antibodies directed to PAK4 can be identified and obtained from a variety of sources, such as, for example, the MSRS Catalog of Primary Antibodies (MSRS/Aerie Corporation, Key West, Fla.), or can be prepared via conventional antibody generation methods.

Detection and analysis of PAK4 expression can be practiced using the inventive antisense oligonucleotides and/or siRNA molecules and a label such as, for example, a radiolabel incorporated into the oligonucleotides by, for example, ³²P labeling at the 5′ end with polynucleotide kinase (Sambrook et al., Molecular Cloning—A Laboratory Manual, Cold Spring Harbor Laboratory Press, Vol. 2, pp. 10-59 (1989)). Radiolabeled oligonucleotides and/or siRNA molecules, for example, can be contacted with tissue or cell samples suspected of PAK4 expression. The sample is then washed to remove unbound oligonucleotide or siRNA. Radioactivity remaining in the sample indicates bound oligonucleotide or siRNA (which in turn indicates the presence of PAK4), which can be quantitated using a scintillation counter or other routine means. Labeled oligonucleotides and/or siRNA molecules can also be used to perform autoradiography of tissues to determine the localization, distribution, and quantitation of PAK4 expression for research, diagnostic, or therapeutic purposes. In such studies, tissue sections are treated with radiolabeled oligonucleotide or siRNA, washed to remove unbound oligonucleotides and/or molecules, and exposed to photographic emulsion according to routine autoradiography procedures. The emulsion, when developed, yields an image of silver grains over the regions expressing PAK4. Quantitation of the silver grains permits PAK4 expression to be detected and analyzed.

Analogous assays for fluorescent detection and analysis of PAK4 expression can be developed using the oligonucleotides and/or siRNA molecules of the invention which are conjugated with fluorescein or other fluorescent tag. Such conjugations are routinely generated in solid phase synthesis using fluorescently labeled amidites or CPG (Glen Research Corp., Sterling, Va.). Alternatively, the cells can be stained using methods well known in the art, such as, for example, propidium iodide staining of DNA, and subsequently detected and analyzed by, for example, flow cytometry.

Pharmaceutical Compositions

Certain embodiments of the present invention include pharmaceutical compositions that comprise the inventive antisense oligonucleotides and/or siRNA molecules as well as other pharmaceutically acceptable compounds, carriers, diluents, agents, salts, enhancers, etc. The additional components in the pharmaceutical compositions may be therapeutic in nature and/or may assist in the uptake, distribution, and/or absorption of the antisense oligonucleotides and/or siRNA molecules.

Pharmaceutical compositions for topical administration include, but are not limited to, transdermal patches, ointments, lotions, creams, gels, drops, suppositories, sprays, liquids, and powders. Conventional pharmaceutical carriers, aqueous, powder, or oily bases, and/or thickeners, can be included in the compositions.

Compositions and formulations for oral administration include, but are not limited to, powders or granules, suspensions or solutions in water or non-aqueous media, capsules, sachets, or tablets. Flavoring agents, diluents, emulsifiers, dispersing aids, and/or binders can be included in the compositions.

Compositions and formulations for parenteral, intrathecal, or intraventricular administration include, but are not limited to, sterile aqueous solutions which optionally contain buffers, diluents, penetration enhancers, carriers, and/or excipients.

The compositions of the present invention may additionally contain components conventionally found in pharmaceutical compositions at their art-established usage levels. Thus, for example, the compositions may contain additional pharmaceutically-active materials such as, for example, antipruritics, astringents, local anesthetics, or anti-inflammatory agents, or may contain additional materials useful in physically formulating various dosage forms of the antisense oligonucleotide-containing compositions, such as, for example, dyes, flavoring agents, preservatives, antioxidants, opacifiers, thickening agents, or stabilizers. However, such materials, when added, should not unduly interfere with the biological activities of the antisense oligonucleotides and/or siRNA molecules.

Combination Treatments

Certain embodiments of the invention provide pharmaceutical compositions containing (a) one or more PAK4 antisense oligonucleotides and/or siRNA molecules, and (b) one or more agents which function by a non-antisense mechanism. Preferably, the agent is effective in treating abnormal cell growth. Examples of such agents include, but are not limited to, chemotherapeutics, mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxic compounds, anti-hormones, anti-androgens, and mixtures thereof.

Anti-inflammatory drugs, including, but not limited to, nonsteroidal anti-inflammatory drugs and corticosteroids, and antiviral drugs may also be combined in compositions of the invention.

As one skilled in the art will appreciate, two or more of the agents may be used together or sequentially. Similarly, two or more combined oligonucleotides and/or siRNA molecules may be used together or sequentially.

In another related embodiment, compositions of the invention contain one or more antisense oligonucleotides and/or siRNA molecules, targeted to a first nucleic acid, and one or more antisense oligonucleotides and/or siRNA molecules targeted to a second nucleic acid. Two or more combined oligonucleotides and/or siRNA molecules may be used together or sequentially.

Dosing

The formulation and subsequent administration of the pharmaceutical compositions can be readily determined by those skilled in the art. Dosing is dependent on severity and responsiveness of the disease or condition to be treated, with the course of treatment lasting from several days to several months, or until a cure is effected or a diminution of the disease or condition is achieved. One of ordinary skill in the art can readily determine optimum dosages, dosing methodologies, and repetition rates. For therapeutics, an animal, preferably, a human, suspected of having a disease or condition which can be diagnosed and/or treated by decreasing or inhibiting the expression of PAK4 is treated by administering antisense oligonucleotides and/or siRNA molecules, or antisense oligonucleotide- and/or siRNA molecule-containing compositions in accordance with the invention.

Without intending to limit the scope of the invention, exemplary methods and their related results, according to various embodiments of the present invention, are given below.

### EXAMPLES

### Example 1

### Antisense Oligonucleotide Preparation

The PAK4 antisense oligonucleotides (Table 1) used in the following examples were commercially synthesized in vitro using standard solid phase synthesis (Integrated DNA Technologies, Coralville, Iowa).

### Example 2

### Cell Culture and Oligonucleotide Treatment

The human lung carcinoma cell line MCF7 was obtained from the American Type Culture Collection (ATCC) (Manassas, Va.). MCF7 cells were cultured in DMEM basal media (Gibco BRL) supplemented with 10% fetal calf serum (Gibco BRL), penicillin (100 units/ml), and streptomycin (100 μg/ml) (Gibco BRL). Cells were passaged by trypsinization and dilution when they reached 90% confluence.

When the cells reached 90% confluence, they were treated with a PAK4 antisense oligonucleotide or control N20 antisense oligonucleotide which is a chemically similar (with respect to linkage, length, and protection groups) sequence-scrambled oligonucleotide comprised of a random stoichiometry of bases at each position, or the cells were not treated (untreated controls (UTC)). The cells, grown in 96-well plates, were treated by washing once with 200 μl Opti-MEM® reduced serum medium (Gibco BRL) and then fed with 130 μl of Opti-MEM® containing 3.75 μg/ml Lipofectin® (Gibco BRL) and the specific concentration (200 nM) of PAK4 antisense oligonucleotide or N20 antisense oligonucleotide. After 4-7 hours, the medium was replaced with fresh medium. Cells were harvested 16-24 hours after oligonucleotide treatment.

### Example 3

### Total RNA Isolation

Total mRNA was isolated using an RNeasy® 96 kit and buffers obtained from Qiagen Inc. (Valencia, Calif.) following the manufacturers instructions. For cells grown in 96-well plates, growth medium was removed from the wells and each well was washed with 200 μl cold PBS (which was subsequently removed). 100 μl buffer RLT were added to each well and the plate vigorously agitated for 20 seconds. 100 μl of 70% ethanol were then added to each well and the contents mixed by pipetting three times up and down. The samples were then transferred to a RNeasy® 96 well plate attached to a QIAvac manifold (Qiagen Inc.) fitted with a waste collection tray and attached to a vacuum source. Vacuum was applied for 15 seconds. Next, 1 ml of buffer RW1 was added to each well of the RNeasy® 96 plate and the vacuum again applied for 15 seconds. Then, 1 ml of buffer RPE was added to each well of the RNeasy® 96 plate and the vacuum again applied for a period of 15 seconds. The buffer RPE wash was repeated and the vacuum applied for an additional 10 minutes. The plate was then removed from the QIAvac manifold and blotted dry on paper towels. The plate was then re-attached to the QIAvac manifold fitted with a collection tube rack containing 1.2 ml collection tubes. RNA was then eluted by pipetting 60 μl water into each well, incubating 1 minute, and then applying the vacuum for 30 seconds. The elution step was repeated with an additional 60 μl of water.

### Example 4

### Real-Time Quantitative PCR Analysis of PAK4 mRNA Levels

Quantitation of PAK4 mRNA levels was determined by real-time quantitative PCR using an ABI PRISM® 7900 Sequence Detection System (Applied Biosystems, Foster City, Calif.) according to manufacturer's instructions. RT-PCR reactions were carried out by adding 10 μl 2×RT-PCR buffer (Applied Biosystems) to 384-well plates containing 10 μl poly(A) total mRNA solution (from Example 3 above). The RT reaction was carried out by incubation for 30 minutes at 48° C. Following a 10-minute incubation at 95° C. to activate the Amplitaq Gold® (in the buffer), 40 cycles of a two-step PCR protocol were carried out: 95° C. for 15 seconds (denaturation) followed by 60° C. for 1.5 minutes (annealing/extension).

Two sets of primers to human PAK4 were designed to hybridize to the human PAK4 sequence set forth in SEQ ID NO:1. Table 3 shows the PCR primers for human PAK4.

TABLE 3

PAK4 PCR Primers & Probes

| | | | |
|---|---|---|---|
| TM | | | Align- |
| (melt- | | | ment |
| ing | | | with SEQ |
| temp | | | ID NO: 1 |
| (° | PAK4 | | (nt res- |
| C.)) | Primers | Sequence (5′-3′) | idues) |

| | | | | |
|---|---|---|---|---|
| SET | 58 | forward | agccatgaagatgattcggg | 1566 |
| A | | primer | (SEQ ID NO: 35) | |
| | 58 | reverse | atggcgacaccttgtgcag | 1630 |
| | | primer | (SEQ ID NO: 36) | |
| | 68 | Taqman ® | caacctgccaccccgactgaaga | 1557 |
| | | probe⁺ | (SEQ ID NO: 37) | |
| SET | 58 | forward | tgggtggtcatggagttcct | 1174 |
| B | | primer | (SEQ ID NO: 38) | |
| | 58 | reverse | tcgttcatcctggtgtgggt | 1241 |
| | | primer | (SEQ ID NO: 39) | |
| | 68 | Taqman ® | aggaggcgccctcaccgacatc | 1197 |
| | | probe⁺ | (SEQ ID NO: 40) | |

⁺labeled with FAM (Applied Biosystems, Foster City, CA) fluorescent reporter dye and TAMRA (Applied Biosystems, Foster City, CA) quencher dye

Antisense inhibition of human PAK4 expression by chimeric phosphorothioate oligonucleotides having 2′-MOE wings and a deoxy gap in accordance with the present invention was tested using a series of oligonucleotides as shown in Table 1 designed to target different regions on the PAK4 mRNA.

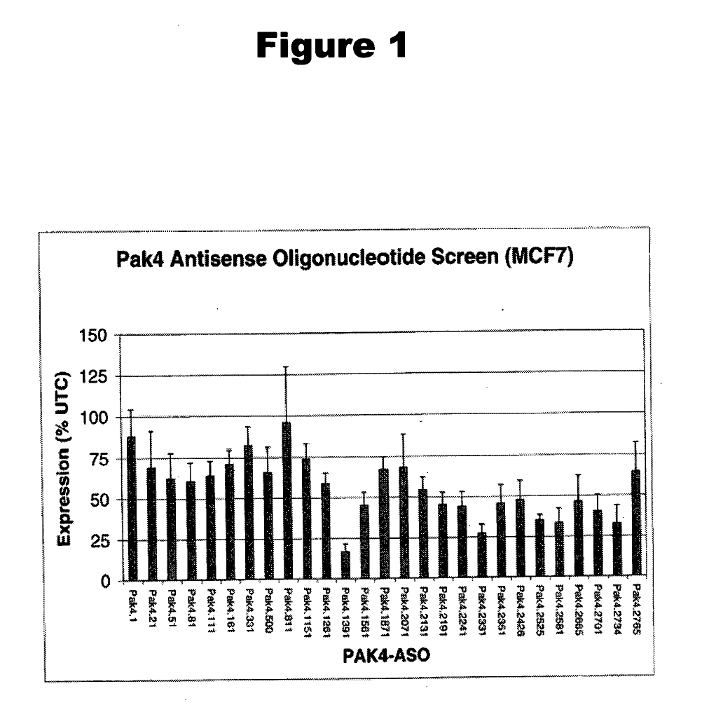

The results are shown in Table 4 and graphically depicted in FIG. 1. Twenty-seven antisense oligonucleotides were screened (as in Example 2 above) for reduction or inhibition of PAK4 expression. Of these, 12 out of 27 demonstrated at least a 50% reduction in PAK4 expression, and 1 out of 27 demonstrated at least a 75% reduction in PAK4 expression.

TABLE 4

PAK4 Antisense Oligonucleotide-induced
Inhibition of PAK4

| | | | | |
|---|---|---|---|---|
| | | Alignment | | Per- |
| | | with | | cent |
| | | SEQ ID | SEQ | Inhi- |
| Position | Sequence | NO: 1 (nt | ID | bition |
| Name | (5′-3′) | residues) | NO: | (%) |

| | | | | |
|---|---|---|---|---|
| PAK4-1 | gacgaattccaccacactgg | 1 | 2 | 12.48 |
| PAK4-21 | cttgcaccgccaccaccgcg | 21 | 3 | 30.87 |
| PAK4-51 | actccgcgccctcgcgcctc | 51 | 4 | 37.78 |
| PAK4-81 | gtcgctcgcggcctaactgc | 81 | 5 | 39.50 |
| PAK4-111 | cttcgggttactcatcggct | 111 | 6 | 36.16 |
| PAK4-161 | atgctggtgggacagaagtg | 161 | 7 | 28.90 |
| PAK4-331 | gccgactcctcgatcaggct | 331 | 8 | 18.08 |
| PAK4-501 | tgtctctccgcagggagttg | 501 | 9 | 34.17 |
| PAK4-811 | gtgttaaagggccggccagc | 811 | 10 | 4.34 |
| PAK4-1151 | gtaggagcgggggtcgcctg | 1151 | 11 | 26.08 |
| PAK4-1261 | tgcttgcgcaggtccatctt | 1261 | 12 | 41.23 |
| PAK4-1391 | tccttccaggaactccatga | 1391 | 13 | 83.31 |
| PAK4-1561 | gacagcttcaccctgccatc | 1561 | 14 | 54.74 |
| PAK4-1871 | ccgctgggcagggtctcgca | 1871 | 15 | 33.12 |
| PAK4-2071 | gcatctcccgggctgggagg | 2071 | 16 | 31.83 |
| PAK4-2131 | aactggagttcagtagtagg | 2131 | 17 | 45.77 |
| PAK4-2191 | tcctgggagcctcgcttgct | 2191 | 18 | 54.78 |
| PAK4-2241 | ttcctggagacagaagaaca | 2241 | 19 | 55.94 |
| PAK4-2331 | gcacacactcatacatgttc | 2331 | 20 | 72.59 |
| PAK4-2351 | acatgcacactcacacgcgt | 2351 | 21 | 54.35 |
| PAK4-2426 | ctgggtgtcaggcaaggcgc | 2426 | 22 | 52.23 |
| PAK4-2525 | tccccatccagccacagaaa | 2525 | 23 | 64.99 |
| PAK4-2581 | aggtgcagtagtcatttgct | 2581 | 24 | 66.74 |
| PAK4-2665 | caggacagggaccatctgtc | 2665 | 25 | 53.48 |
| PAK4-2701 | gcagtggttctgccagggcc | 2701 | 26 | 59.86 |
| PAK4-2734 | ctgcgctgaccgggcaggaa | 2734 | 27 | 67.21 |
| PAK4-2765 | ctaactcgaggcaggggtgg | 2765 | 28 | 35.58 |

### Example 5

### siRNA Preparation

The PAK4 RNA-interfering siRNA molecules used in the following examples were commercially synthesized in vitro via “ready to use mode C” (Dharmacon, Inc., Lafayette, Colo.). The siRNAs were chemically synthesized using appropriately protected ribonucleoside phosphoramidites. Table 2 lists the siRNA interfering molecules that were tested in the following examples.

### Example 6

### Transfection of siRNA Molecules

Cultured A549 cells were transfected with the siRNA molecules using standard techniques available commercially (GeneSilencer™, Gene Therapy Systems, Inc., San Diego, Calif.). The manufacturer's instructions set forth procedures for 6-well, 24-well, 48-well, and 96-well plates.

The day before transfection, adherent cells were plated for 50-70% confluency on the day of transfection. The GeneSilencer™ reagent was prepared by diluting it in serum-free medium such that, for a 6-well plate, 5.0 μl reagent was diluted in 25 μl medium. The siRNA solution was prepared by first mixing 25 μl siRNA diluent and 15 μl serum-free medium and then adding 1000 ng siRNA per well. The solution was mixed by pipetting it up and down several times. The solution was then incubated at room temperature for 5 minutes.

The siRNA solution was then added to the diluted GeneSilencer™ reagent and incubated at room temperature for 5 minutes to allow siRNA/lipid complexes to form. The siRNA/GeneSilencer™ mix was then added to cells growing in serum-containing medium and incubated at 37° C. for 24 hours. Fresh tissue culture medium was added to the growing cells as needed. Most of the RNA interference was detected within 24 to 72 hours post-transfection.

### Example 7

### Total RNA Isolation

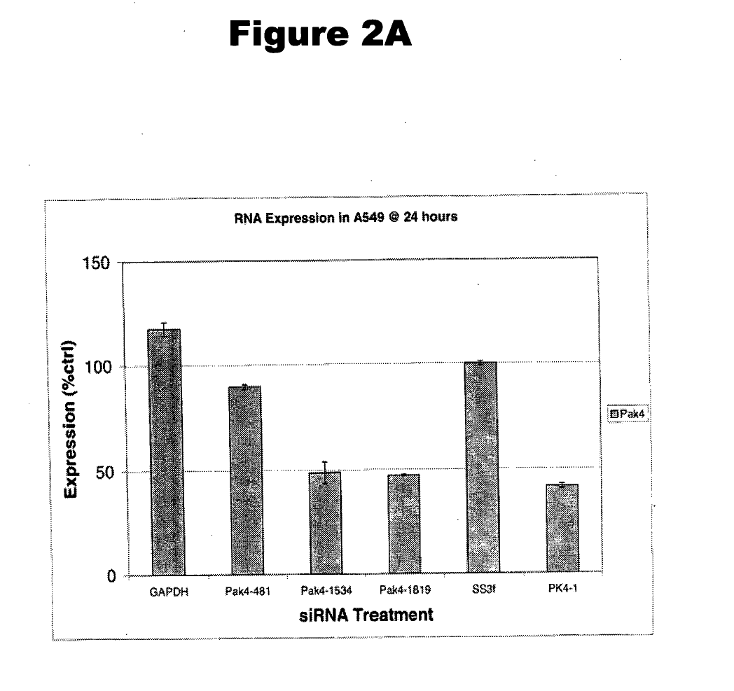

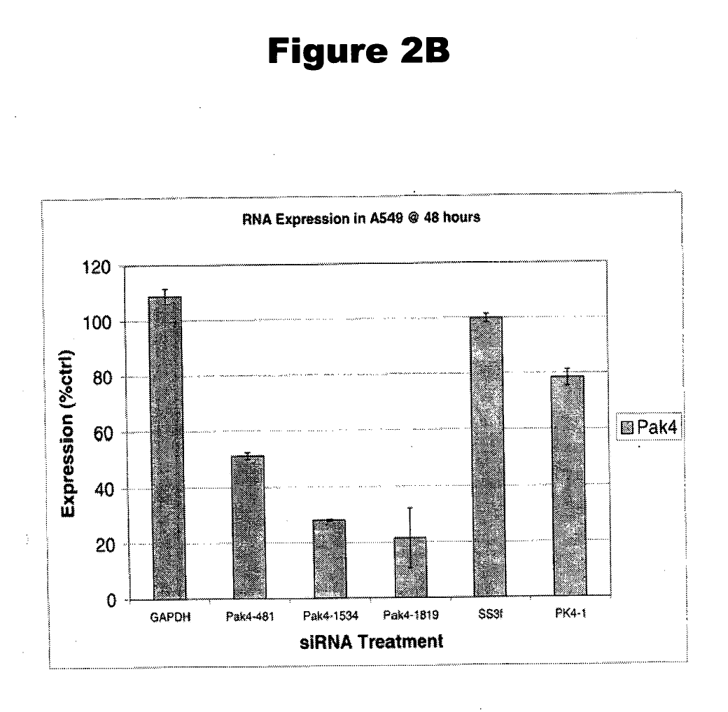

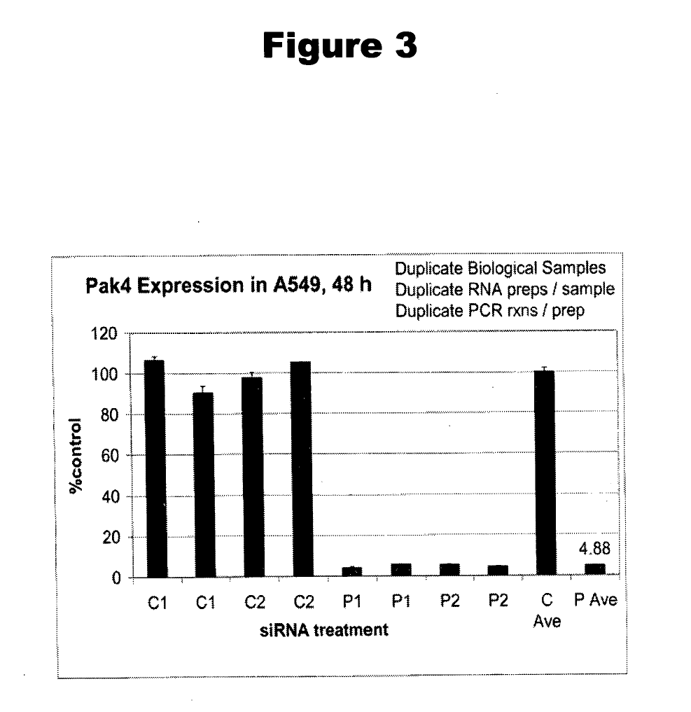

RNA expression was determined as set forth above in Example 3. FIG. 2 shows PAK4 expression 24 hours (A) and 48 hours (B) following treatment with three siRNA molecules. Control cells were treated with GAPDH siRNA (Ambion, Inc., Austin, Tex.) (specific for glyceraldehyde-3-phosphate dehydrogenase, included as a positive control (immunoblotting or performing RT-PCR for GAPDH protein or mRNA, respectively)); SS3f siRNA (artificial non-specific sequence, included as a control for assessing the effects of transfection on cell stability and health) (sense strand 5′-3′: fluorescein-ugaccucuagcuaccacagtt (SEQ ID NO:47) and antisense strand 5′-3′: cugugguagcuagaggucatt (SEQ ID NO:48)); and PK4-1 siRNA. At 24 hours, treatment with PAK4-1534 and PAK4-1819 had reduced PAK4 expression by at least 50% of the GAPDH and SS3f controls (FIG. 2A). By 48 hours, PAK4 expression was reduced to 30% of the same controls by the same siRNA molecules (FIG. 2B). FIG. 3 describes an experiment with ‘optimized’ cell confluency; this was the result of improvements in the treatment and conditions of the experiment FIG. 2 (70% confluent cell culture) and FIG. 3 (85-90% confluent cell culture).

While the invention has been illustrated by reference to specific and preferred embodiments, those skilled in the art will recognize that variations and modifications may be made through routine experimentation and practice of the invention. Thus, the invention is intended not to be limited by the foregoing description, but to be defined by the appended claims and their equivalents.

Sequences

| | |
|---|---|
| SEQ ID NO: 1 = | GenBank Accession No. AF005046 |
| | (PAK4 nucleotide sequence) |
| SEQ ID NO: 2 = | gacgaattccaccacactgg (PAK4-1) |
| SEQ ID NO: 3 = | cttgcaccgccaccaccgcg (PAK4-21) |
| SEQ ID NO: 4 = | actccgcgccctcgcgcctc (PAK4-51) |
| SEQ ID NO: 5 = | gtcgctcgcggcctaactgc (PAK4-81) |
| SEQ ID NO: 6 = | cttcgggttactcatcggct (PAK4-111) |
| SEQ ID NO: 7 = | atgctggtgggacagaagtg (PAK4-161) |
| SEQ ID NO: 8 = | gccgactcctcgatcaggct (PAK4-331) |
| SEQ ID NO: 9 = | tgtctctccgcagggagttg (PAK4-501) |
| SEQ ID NO: 10 = | gtgttaaagggccggccagc (PAK4-811) |
| SEQ ID NO: 11 = | gtaggagcgggggtcgcctg (PAK4-1151) |
| SEQ ID NO: 12 = | tgcttgcgcaggtccatctt (PAK4-1261) |
| SEQ ID NO: 13 = | tccttccaggaactccatga (PAK4-1391) |
| SEQ ID NO: 14 = | gacagcttcaccctgccatc (PAK4-1561) |
| SEQ ID NO: 15 = | ccgctgggcagggtctcgca (PAK4-1871) |
| SEQ ID NO: 16 = | gcatctcccgggctgggagg (PAK4-2071) |
| SEQ ID NO: 17 = | aactggagttcagtagtagg (PAK4-2131) |
| SEQ ID NO: 18 = | tcctgggagcctcgcttgct (PAK4-2191) |
| SEQ ID NO: 19 = | ttcctggagacagaagaaca (PAK4-21) |
| SEQ ID NO: 20 = | gcacacactcatacatgttc (PAK4-2241) |
| SEQ ID NO: 21 = | acatgcacactcacacgcgt (PAK4-2331) |
| SEQ ID NO: 22 = | ctgggtgtcaggcaaggcgc (PAK4-2351) |
| SEQ ID NO: 23 = | tccccatccagccacagaaa (PAK4-2426) |
| SEQ ID NO: 24 = | aggtgcagtagtcatttgct (PAK4-2525) |
| SEQ ID NO: 25 = | caggacagggaccatctgtc (PAK4-2581) |
| SEQ ID NO: 26 = | gcagtggttctgccagggcc (PAK4-2665) |
| SEQ ID NO: 27 = | ctgcgctgaccgggcaggaa (PAK4-2701) |
| SEQ ID NO: 28 = | ctaactcgaggcaggggtgg (PAK4-2734) |
| SEQ ID NO: 29 = | caugucggugacacgcucctt (PAK4-481 |
| | sense RNA strand) |
| SEQ ID NO: 30 = | ggagcgugucaccgacaugtt (PAK4-481 |
| | antisense) |
| SEQ ID NO: 31 = | gagcgacucgauccugcugtt (PAK4-1534 |
| | sense RNA strand) |
| SEQ ID NO: 32 = | cagcaggaucgagucgcuctt (PAK4-1534 |
| | antisense) |
| SEQ ID NO: 33 = | ccugcacaaggugucgccatt (PAK4-1819 |
| | sense RNA strand) |
| SEQ ID NO: 34 = | uggcgacaccuugugcaggtt (PAK4-1819 |
| | antisense) |
| SEQ ID NO: 35 = | agccatgaagatgattcggg (set A - PAK4 |
| | forward primer) |
| SEQ ID NO: 36 = | atggcgacaccttgtgcag (set A - PAK4 |
| | reverse primer) |
| SEQ ID NO: 37 = | caacctgccaccccgactgaaga (set A - |
| | PAK4 Taqman ® probe) |
| SEQ ID NO: 38 = | tgggtggtcatggagttcct (set B - PAK4 |
| | forward primer) |
| SEQ ID NO: 39 = | tcgttcatcctggtgtgggt (set B - PAK4 |
| | reverse primer) |
| SEQ ID NO: 40 = | aggaggcgccctcaccgacatc (set B - |
| | PAK4 Taqman ® probe) |
| SEQ ID NO: 41 = | aacatgtcggtgacacgctcc (PAK4-481 |
| | target) |
| SEQ ID NO: 42 = | aagagcgactcgatcctgctg (PAK4-1534 |
| | target) |
| SEQ ID NO: 43 = | aacctgcacaaggtgtcgcca (PAK4-1819 |
| | target) |
| SEQ ID NO: 44 = | aactcgccaatcttgatgaag (PAK4-1 |
| | target) |
| SEQ ID NO: 45 = | cuucaucaagauuggcgagtt (PAK4-1 |
| | sense RNA strand) |
| SEQ ID NO: 46 = | cucgccaaucuugaugaagtt (PAK4-1 |
| | antisense) |
| SEQ ID NO: 47 = | tgacctctagctaccacagtt (SS3f sense |
| | strand) |
| SEQ ID NO: 48 = | cugugguagcuagaggucatt (SS3f |
| | antisense) |

1. An antisense oligonucleotide comprising from about 8 to about 50 nucleic acid bases in length targeted to a nucleic acid molecule encoding PAK4, wherein said antisense oligonucleotide decreases or inhibits the expression of PAK4.

2. The antisense oligonucleotide of claim 1, wherein PAK4 is human PAK4 having the nucleotide sequence of SEQ ID NO:1.

3. The antisense oligonucleotide of claim 1, consisting of about 8 to about 50 nucleic acid bases.

4. The antisense oligonucleotide of claim 3, consisting of about 8 to about 30 nucleic acid bases.

5. The antisense oligonucleotide of claim 4, having a sequence selected from the group consisting of: SEQ ID NOS:2, 3, 4, 5, 6, 7, 8, 9, 10, 11, 12, 13, 14, 15, 16, 17, 18, 19, 20, 21, 22, 23, 24, 25, 26, 27, and 28.

6. The antisense oligonucleotide of claim 1, comprising at least one modified internucleoside linkage.

7. The antisense oligonucleotide of claim 6, wherein the modified internucleoside linkage is a phosphorothioate linkage.

8. The antisense oligonucleotide of claim 1, comprising at least one modified sugar moiety.

9. The antisense oligonucleotide of claim 8, wherein the modified sugar moiety is a 2′-O-methoxymethyl sugar moiety.

10. The antisense oligonucleotide of claim 1, comprising at least one modified nucleic acid base.

11. The antisense oligonucleotide of claim 10, wherein the modified nucleic acid base is 5-methylcytosine.

12. A pharmaceutical composition comprising the antisense oligonucleotide of claim 1 and a pharmaceutically acceptable carrier, diluent, or salt.

13. The pharmaceutical composition of claim 12 further comprising an antisense oligonucleotide targeted to a nucleic acid molecule that does not encode PAK4.

14. The pharmaceutical composition of claim 12 further comprising a therapeutic agent, wherein said agent is not an antisense oligonucleotide.

15. The pharmaceutical composition of claim 14, wherein the agent is selected from the group consisting of: mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxic compounds, anti-hormones, and anti-androgens, and RNA-interfering nucleic acid sequences.

16. A method of decreasing or inhibiting the expression of PAK4 in cells or tissues comprising contacting said cells or tissues with the antisense oligonucleotide of claim 1 so that expression of PAK4 is decreased or inhibited.

17. The method of claim 16, wherein said cells or tissues are human cells or tissues.

18. A method of treating a human having or suspected of having a disease or condition associated with PAK4 expression comprising administering to said human a therapeutically effective amount of the pharmaceutical composition of claim 12 so that expression of PAK4 is decreased or inhibited.

19. The method of claim 18, wherein said disease or condition is abnormal cell growth.

20. The method of claim 19, wherein the abnormal cell growth is cancer.

21. The method of claim 19, wherein the abnormal cell growth is benign proliferative disease.

22. The method of claim 19, wherein the abnormal cell growth is selected from the group consisting of: psoriasis, benign prostatic hypertrophy, and restinosis.

23. The method of claim 18, wherein the disease or condition is an inflammatory disease or condition.

24. The method of claim 23, wherein the inflammatory disease or condition is an autoimmune disease, cell-mediated rejection, graft-versus-host disease, or arthritis.

25. A ribonucleic acid (RNA)-interfering molecule comprising a double-stranded RNA molecule with a first strand comprising a ribonucleotide sequence which corresponds to a nucleotide sequence encoding PAK4 and a second strand comprising a ribonucleotide sequence which is complementary to a nucleotide sequence encoding PAK4, wherein the first and second ribonucleotide strands are separate complementary strands that hybridize to each other to form said double-stranded RNA molecule, and wherein the double-stranded RNA molecule decreases or inhibits the expression of PAK4.

26. The antisense oligonucleotide of claim 25 wherein PAK4 is human PAK4 having the nucleotide sequence of SEQ ID NO:1.

27. The RNA-interfering molecule of claim 25, wherein the first ribonucleotide sequence comprises from about 8 to about 30 nucleic acid bases which correspond to a nucleotide sequence encoding PAK4 and the second ribonucleotide sequence comprises from about 8 to about 30 nucleic acid bases which are complementary to the nucleotide sequence encoding PAK4.

28. The RNA-interfering molecule of claim 27, wherein the first and second ribonucleotide sequences are each about 21 nucleotides in length.

29. The RNA-interfering molecule of claim 28, wherein the first nucleotide sequence is a sequence selected from the group consisting of: SEQ ID NOS:29, 31, and 33, and wherein the second nucleotide sequence is a sequence selected from the group consisting of: SEQ ID NOS:30, 32, and 34.

30. A pharmaceutical composition comprising the RNA-interfering molecule of claim 25 and a pharmaceutically acceptable carrier, diluent, or salt.

31. The pharmaceutical composition of claim 30, further comprising an RNA-interfering molecule targeted to a nucleotide sequence that does not encode PAK4.

32. The pharmaceutical composition of claim 30, further comprising a therapeutic agent, wherein said agent is not an RNA-interfering molecule.

33. The pharmaceutical composition of claim 32, wherein the agent is selected from the group consisting of: mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxic compounds, anti-hormones, anti-androgens, and antisense oligonucleotides.

34. A method of decreasing or inhibiting the expression of PAK4 in cells or tissues comprising contacting said cells or tissues with the RNA-interfering molecule of claim 25 so that expression of PAK4 is decreased or inhibited.

35. The method of claim 34, wherein said cells or tissues are human cells or tissues.

36. A method of treating a human having or suspected of having a disease or condition associated with PAK4 expression, comprising administering to said human a therapeutically effective amount of the pharmaceutical composition of claim 30 so that expression of PAK4 is decreased or inhibited.

37. The method of claim 36, wherein said disease or condition is abnormal cell growth.

38. The method of claim 37, wherein the abnormal cell growth is cancer.

39. The method of claim 37, wherein the abnormal cell growth is benign proliferative disease.

40. The method of claim 37, wherein the abnormal cell growth is selected from the group consisting of: psoriasis, benign prostatic hypertrophy, and restinosis.

41. The method of claim 36, wherein the disease or condition is an inflammatory disease or condition.

42. The method of claim 41, wherein the inflammatory disease or condition is an autoimmune disease, cell-mediated rejection, graft-versus-host disease, or arthritis.


SEQUENCE LISTING (48 sequences)

SEQ ID NO: 1 - DNA, 2838 bp, Homo sapiens
ccagtgtggtggaattcgtccgcggtggtggcggtgcaagagagctgagggaggcgcgagggcgcggagttccaggtcgagcagttaggccgcgagcgactgcggcgccgagccgatgagtaacccgaagcccctagaggagtggtcacctgcctgagggcacttctgtcccaccagcatcagaccaggccgcaccgagtccccggcaccatgtttgggaagaggaagaagcgggtggagatctccgcgccgtccaacttcgagcaccgcgtgcacacgggcttcgaccagcacgagcagaagttcacggggctgccccgccagtggcagagcctgatcgaggagtcggctcgccggcccaagcccctcgtcgaccccgcctgcatcacctccatccagcccggggcccccaagaccatcgtgcggggcagcaaaggtgccaaagatggggccctcacgctgctgctggacgagtttgagaacatgtcggtgacacgctccaactccctgcggagagacagcccgccgccgcccgcccgtgcccgccaggaaaatgggatgccagaggagccggccaccacggccagagggggcccagggaaggcaggcagccgaggccggttcgccggtcacagcgaggcgggtggcggcagtggtgacaggcgacgggcggggccagagaagaggcccaagtcttccagggagggctcagggggtccccaggagtcctcccgggacaaacgccccctctccgggcctgatgtcggcaccccccagcctgctggtctggccagtggggcgaaactggcagctggccggccctttaacacctacccgagggctgacacggaccacccatcccggggtgcccagggggagcctcatgacgtggcccctaacgggccatcagcggggggcctggccatcccccagtcctcctcctcctcctcccggcctcccacccgagcccgaggtgcccccagccctggagtgctgggaccccacgcctcagagccccagctggcccctccagcctgcacccccgccgcccctgctgttcctgggccccctggcccccgctcaccacagcgggagccacagcgagtatcccatgagcagttccgggctgccctgcagctggtggtggacccaggcgacccccgctcctacctggacaacttcatcaagattggcgagggctccacgggcatcgtgtgcatcgccaccgtgcgcagctcgggcaagctggtggccgtcaagaagatggacctgcgcaagcagcagaggcgcgagctgctcttcaacgaggtggtaatcatgagggactaccagcacgagaatgtggtggagatgtacaacagctacctggtgggggacgagctctgggtggtcatggagttcctggaaggaggcgccctcaccgacatcgtcacccacaccaggatgaacgaggagcagatcgcggccgtgtgccttgcagtgctgcaggccctgtcggtgctccacgcccagggcgtcatccaccgggacatcaagagcgactcgatcctgctgacccatgatggcagggtgaagctgtcagactttgggttctgcgcccaggtgagcaaggaagtgccccgaaggaagtcgctggtcggcacgccctactggatggccccagagctcatctcccgccttccctacgggccagaggtagacatctggtcgctggggataatggtgattgagatggtggacggagagcccccctacttcaacgagccacccctcaaagccatgaagatgattcgggacaacctgccaccccgactgaagaacctgcacaaggtgtcgccatccctgaagggcttcctggaccgcctgctggtgcgagaccctgcccagcgggccacggcagccgagctgctgaagcacccattcctggccaaggcagggccgcctgccagcatcgtgcccctcatgcgccagaaccgcaccagatgaggcccagcgcccttcccctcaaccaaagagccccccgggtcacccccgccccactgaggccagtagggggccaggcctcccactcctcccagcccgggagatgctccgcgtggcaccaccctccttgctgggggtagatgagaccctactactgaactccagttttgatctcgtgacttttagaaaaacacagggactcgtgggagcaagcgaggctcccaggacccccaccctctgggacaggccctcccccatgttcttctgtctccaggaagggcagcggccctcccatcactggaagtctgcagtgggggtcgctgggggtggagagaacactaagaggtgaacatgtatgagtgtgtgcacgcgtgtgagtgtgcatgtgtgtgtgtgtgcaaaggtccagccaccccgtcctccagcccgcaaggggtgtctggcgccttgcctgacacccagccccctctccccctgagccattgtgggggtcgatcatgaatgtccgaagagtggccttttcccgtagccctgcgccccctttctgtggctggatggggagacaggtcagggccccccaccctctccagcccctgcagcaaatgactactgcacctggacagcctcctcttttctagaagtctatttatattgtcattttataacactctagcccctgcccttattgggggacagatggtccctgtcctgcggggtggccctggcagaaccactgcctgaagaaccaggttcctgcccggtcagcgcagccccagcccgcccacccctgcctcgagttagttttacaattaaaacattgtcttgtttaaaaaaaaaaaaaaaaaaaaaaaaaaa

SEQ ID NO: 2 - DNA, 20 bp, Homo sapiens
gacgaattccaccacactgg

SEQ ID NO: 3 - DNA, 20 bp, Homo sapiens
cttgcaccgccaccaccgcg

SEQ ID NO: 4 - DNA, 20 bp, Homo sapiens
actccgcgccctcgcgcctc

SEQ ID NO: 5 - DNA, 20 bp, Homo sapiens
gtcgctcgcggcctaactgc

SEQ ID NO: 6 - DNA, 20 bp, Homo sapiens
cttcgggttactcatcggct

SEQ ID NO: 7 - DNA, 20 bp, Homo sapiens
atgctggtgggacagaagtg

SEQ ID NO: 8 - DNA, 20 bp, Homo sapiens
gccgactcctcgatcaggct

SEQ ID NO: 9 - DNA, 20 bp, Homo sapiens
tgtctctccgcagggagttg

SEQ ID NO: 10 - DNA, 20 bp, Homo sapiens
gtgttaaagggccggccagc

SEQ ID NO: 11 - DNA, 20 bp, Homo sapiens
gtaggagcgggggtcgcctg

SEQ ID NO: 12 - DNA, 20 bp, Homo sapiens
tgcttgcgcaggtccatctt

SEQ ID NO: 13 - DNA, 20 bp, Homo sapiens
tccttccaggaactccatga

SEQ ID NO: 14 - DNA, 20 bp, Homo sapiens
gacagcttcaccctgccatc

SEQ ID NO: 15 - DNA, 20 bp, Homo sapiens
ccgctgggcagggtctcgca

SEQ ID NO: 16 - DNA, 20 bp, Homo sapiens
gcatctcccgggctgggagg

SEQ ID NO: 17 - DNA, 20 bp, Homo sapiens
aactggagttcagtagtagg

SEQ ID NO: 18 - DNA, 20 bp, Homo sapiens
tcctgggagcctcgcttgct

SEQ ID NO: 19 - DNA, 20 bp, Homo sapiens
ttcctggagacagaagaaca

SEQ ID NO: 20 - DNA, 20 bp, Homo sapiens
gcacacactcatacatgttc

SEQ ID NO: 21 - DNA, 20 bp, Homo sapiens
acatgcacactcacacgcgt

SEQ ID NO: 22 - DNA, 20 bp, Homo sapiens
ctgggtgtcaggcaaggcgc

SEQ ID NO: 23 - DNA, 20 bp, Homo sapiens
tccccatccagccacagaaa

SEQ ID NO: 24 - DNA, 20 bp, Homo sapiens
aggtgcagtagtcatttgct

SEQ ID NO: 25 - DNA, 20 bp, Homo sapiens
caggacagggaccatctgtc

SEQ ID NO: 26 - DNA, 20 bp, Homo sapiens
gcagtggttctgccagggcc

SEQ ID NO: 27 - DNA, 20 bp, Homo sapiens
ctgcgctgaccgggcaggaa

SEQ ID NO: 28 - DNA, 20 bp, Homo sapiens
ctaactcgaggcaggggtgg

SEQ ID NO: 29 - DNA, 21 bp, Homo sapiens
caugucggugacacgcucctt

SEQ ID NO: 30 - DNA, 21 bp, Homo sapiens
ggagcgugucaccgacaugtt

SEQ ID NO: 31 - DNA, 21 bp, Homo sapiens
gagcgacucgauccugcugtt

SEQ ID NO: 32 - DNA, 21 bp, Homo sapiens
cagcaggaucgagucgcuctt

SEQ ID NO: 33 - DNA, 21 bp, Homo sapiens
ccugcacaaggugucgccatt

SEQ ID NO: 34 - DNA, 21 bp, Homo sapiens
uggcgacaccuugugcaggtt

SEQ ID NO: 35 - DNA, 20 bp, Homo sapiens
agccatgaagatgattcggg

SEQ ID NO: 36 - DNA, 19 bp, Homo sapiens
atggcgacaccttgtgcag

SEQ ID NO: 37 - DNA, 23 bp, Homo sapiens
caacctgccaccccgactgaaga

SEQ ID NO: 38 - DNA, 20 bp, Homo sapiens
tgggtggtcatggagttcct

SEQ ID NO: 39 - DNA, 20 bp, Homo sapiens
tcgttcatcctggtgtgggt

SEQ ID NO: 40 - DNA, 22 bp, Homo sapiens
aggaggcgccctcaccgacatc

SEQ ID NO: 41 - DNA, 21 bp, Homo sapiens
aacatgtcggtgacacgctcc

SEQ ID NO: 42 - DNA, 21 bp, Homo sapiens
aagagcgactcgatcctgctg

SEQ ID NO: 43 - DNA, 21 bp, Homo sapiens
aacctgcacaaggtgtcgcca

SEQ ID NO: 44 - DNA, 21 bp, Homo sapiens
aactcgccaatcttgatgaag

SEQ ID NO: 45 - DNA, 21 bp, Homo sapiens
cuucaucaagauuggcgagtt

SEQ ID NO: 46 - DNA, 21 bp, Homo sapiens
cucgccaaucuugaugaagtt

SEQ ID NO: 47 - DNA, 21 bp, Homo sapiens
tgacctctagctaccacagtt

SEQ ID NO: 48 - DNA, 21 bp, Homo sapiens
cugugguagcuagaggucatt